SEQUENCE LISTING (12 sequences)

SEQ ID NO: 1 - protein, 7 aa, Artificial Sequence
  Synthetic
SSSYYWG

SEQ ID NO: 2 - protein, 16 aa, Artificial Sequence
  Synthetic
SFFYTGSTYYNPSLRS

SEQ ID NO: 3 - protein, 17 aa, Artificial Sequence
  Synthetic
QSTYYYGSGNYYGWFDR

SEQ ID NO: 4 - protein, 11 aa, Artificial Sequence
  Synthetic
RASQSVSSYLA

SEQ ID NO: 5 - protein, 7 aa, Artificial Sequence
  Synthetic
DASNRAT

SEQ ID NO: 6 - protein, 9 aa, Artificial Sequence
  Synthetic
QQRSNWPPA

SEQ ID NO: 7 - protein, 127 aa, Artificial Sequence
  Synthetic
QLQLQESGPGLVKPSETLSLTCTVSGGSINSSSYYWGWLRQSPGKGLEWIGSFFYTGSTYYNPSLRSRLTISVDTSKNQFSLMLSSVTAADTAVYYCARQSTYYYGSGNYYGWFDRWDQGTLVTVSS

SEQ ID NO: 8 - protein, 107 aa, Artificial Sequence
  Synthetic
EIVLTQSPATLSLSPGERATLSCRASQSVSSYLAWYQQKPGQAPRLLIYDASNRATGIPARFSGSGSGTDFTLTISSLEPEDFAVYYCQQRSNWPPAFGQGTKVEIK

SEQ ID NO: 9 - protein, 476 aa, Artificial Sequence
  Synthetic
MGWSCIILFLVATATGVHSQLQLQESGPGLVKPSETLSLTCTVSGGSINSSSYYWGWLRQSPGKGLEWIGSFFYTGSTYYNPSLRSRLTISVDTSKNQFSLMLSSVTAADTAVYYCARQSTYYYGSGNYYGWFDRWDQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 233 aa, Artificial Sequence
  Synthetic
MGWSCIILFLVATATGVHSEIVLTQSPATLSLSPGERATLSCRASQSVSSYLAWYQQKPGQAPRLLIYDASNRATGIPARFSGSGSGTDFTLTISSLEPEDFAVYYCQQRSNWPPAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - DNA, 1431 bp, Artificial Sequence
  Synthetic
atgggatggtcatgtatcatcctttttctagtagcaactgcaactggagtacattcacagctgcagctgcaggagtcgggcccaggactggtgaagccttcggagaccctgtccctcacctgcactgtctctggtggctccatcaacagtagtagttactactggggctggctccgccagtccccagggaaggggctggagtggattgggagtttcttttatactgggagcacctactacaacccgtccctcaggagtcgactcaccatatccgtagacacgtccaagaaccagttctccctgatgctgagttctgtgaccgccgcagacacggctgtatattactgtgcgagacagtccacgtattactatggttcggggaattattatggctggttcgaccgctgggaccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 12 - DNA, 702 bp, Artificial Sequence
  Synthetic
atgggatggtcatgtatcatcctttttctagtagcaactgcaactggagtacattcagaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccggcgttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

(19) United States
(12) Patent Application Publication
(10) Pub. No.: US 2017/0129958 A1
(43) Pub. Date: May 11, 2017

(54) COMBINATION THERAPY

(71) Applicant: ImClone LLC, Indianapolis, IN (US)

(72) Inventors: Nick LOIZOS, Closter, NJ (US); Gaurav D. SHAH, New York, NY (US)

(73) Assignee: ImClone LLC, Indianapolis, IN (US)

(21) Appl. No.: 15/318,370

(22) PCT Filed: Jun. 26, 2015
(86) PCT No.: PCT/US2015/037892

§ 371 Date: Dec. 13, 2016

Related U.S. Application Data
(60) Provisional application No. 62/020,427, filed on Jul. 3, 2014.

(51) Int. Cl. C07K 16/28; A61K 31/496; A61K 45/06; A61K 39/395; A61K 31/704

(52) U.S. Cl. CPC C07K 16/2863; A61K 39/39558; A61K 31/704; A61K 45/06; A61K 31/496; C07K 2317/21; A61K 2039/505

(57) ABSTRACT

Combination of human antibodies that bind to human platelet-derived growth factor receptor alpha (PDGFR alpha), preferably olaratumab, and doxorubicin as a medicament for the treatment of soft tissue sarcoma.

This application claims the benefit of U.S. Provisional Application No. 62/020,427 which was filed 3 Jul. 2014.

This invention is directed to the fields of immunology and cancer treatment. More specifically, the present invention is directed to the combination of olaratumab and doxorubicin, and to methods of use of the combination to treat soft tissue sarcoma or as a medicament for the treatment of soft tissue sarcoma.

Soft tissue sarcoma (STS) originates in soft or connective tissue like fat, muscle, nerves, fibrous tissues, blood vessels, or deep skin tissues; all of mesenchymal origin. STS is a heterogeneous disease for which there are relatively few effective regimens, with about 11,400 new cases and about 4400 deaths per year in the U.S. A Snapshot of Sarcoma, National Cancer Institute, US Dept. of Health and Human Services, National Institutes of Health, posted online Dec. 2, 2013 at http://www.cancer.gov/researchandfunding/snapshots/sarcoma. STS includes a variety of subtypes; the prevalence of some of the various subtypes is extremely rare. Despite differences in tissue derivation of sarcomas, these tumors tend to share many similarities: they are often classified generally as STS for the purposes of identifying and prescribing treatment. Accordingly, for the purpose herein, the subtypes are collectively labeled as STS.

The present invention seeks to provide a response to a clinically unmet need for treatment of STS. In this regard, olaratumab plus doxorubicin for the treatment of STS provides unexpected statistically significant survival benefit. Few therapeutic approaches are currently available to patients with advanced STS. Chemotherapy in this setting is essentially palliative in intent; doxorubicin is the standard of care for the majority of such patients, with an associated response rate of 10%-30%.

Doxorubicin is a cytotoxic antibiotic of the anthracyline family of compounds. Its cytotoxic effects are believed to stem from intercalation with DNA nucleotides leading to inactivation of topoisomerase II DNA repair, and generation of free radicals leading to lipid peroxidation and cell membrane damage. Investigations of cellular exposure to doxorubicin have shown morphological changes associated with apoptosis.

Recent and ongoing STS trials have investigated a variety of therapies including combination chemotherapeutic regimens, with and without doxorubicin; however, although selected trials have demonstrated improvements in response rate, there has been little improvement in survival. See Benjamin RS, et al., Med Pediatr Oncol. 1975; 1(1):63-76 (discussing doxorubicin monotherapy); Bramwell V, et. al., Cochrane Database of Systematic Reviews 2001, Issue 4. Art. No.: CD003293. DOI: 10.1002/14651858.CD003293. Cochrane Database of Systematic & Reviews, Issue 4, 2009 (Status in this issue: Unchanged) (discussing monotherapy versus combination doxorubicin); Mouridsen H T, et al., Eur. J. of Cancer and Clin. Onc. 23(10):1477-1483 (1987) (discussing monotherapy doxorubicin versus epirubicin monotherapy); Lorigan P, et al., J Clin Oncol. 2007; 25(21):3144-3150 (discussing doxorubicin versus ifosfamide); Leyvraz S, et al., Br J Cancer. 2006;95(10):1342-1347 (discussing doxorubicin versus ifosfamide); Judson I, et al., Lancet Online. Mar. 5, 2014; http://dx.doi.org/10.1016/S1470-2045(14)70063-4 (discussing doxorubicin versus ifosfamide); Edmonson J H, et al., J Clin Oncol. 1993; 11:1269-1275 (discussing doxorubicin versus doxorubicin/ifosfamide and doxorubicin/mitomycin/cisplatin); Schoenfeld D A, et al., Cancer. 1982; 50:2757-2762 (discussing doxorubicin versus the combination of vincristine plus actinomycin-D plus cyclophosphamide). Recent phase 2 studies involving tyrosine kinase inhibitors (TKIs) have also shown limited success. See Kasper B, et al. Ann Oncol 2014. Published online: Feb. 6, 2014 at http://annonc.oxfordjournals.org/content/early/2014/02/05/annonc.mdt586.abstract (discussing pazopanib); Maki R G, et al., J Clin Oncol. 2009;27(19):3133-3140 (discussing sorafenib); (Chugh R, et al., J Clin Oncol. 2009; 27(19):3148-3153 (discussing imatinib); George S, et al., J Clin Oncol. 2009; 27(19):3154-3160 (discussing sunitinib). In short, there is a high unmet clinical need for new treatments for advanced STS that provide a survival benefit. The present invention seeks to provide a new treatment to meet this need.

A novel combination of olaratumab and doxorubicin for the treatment of STS is herein presented. Olaratumab, IMC-3G3 (U.S. Pat. Nos 8,128,929 and 8,574,578), is a recombinant human monoclonal antibody that specifically targets the human platelet-derived growth factor receptor alpha (PDGFRα or PDGFR alpha). The patents disclose the treatment of a variety of neoplastic diseases, including soft tissue sarcomas, with PDGFRα antibodies, including IMC-3G3. Combination therapies are referenced therein.

Drug development is unpredictable. Not all drugs are equally active in various disease states. It's common for new molecules to fail in preclinical and/or clinical stages, often for little understood reasons. Olaratumab has not shown success in some of its clinical trials to date, including advanced non-small cell lung cancer. Gerber D. et. al. J Clin Oncol 32:5s, 2014 (suppl; abstr 8050) (discussing “A randomized phase 2 study of a human antiplatelet-derived growth factor a (PDGFRa) monoclonal antibody (olaratumab, IMC-3G3) with paclitaxel/carboplatin or paclitaxel/carboplatin alone in previously untreated patients with advanced non-small cell lung cancer (NSCLC)” http://meetinglibrary.asco.org/content/134011-144).

The present invention was studied in a combination Phase 1b and randomized Phase 2 trial (http://www.clinicaltrials.gov/ct2/show/NCT01185964?term=IMC-3G3&rank=3) (hereinafter “Study”). As described therein, the Study enrolled patients with a multitude of subtypes of STS that were stratified at Study entry; the patient population, with respect to the subtypes represented in the Study, was determined to be representative of the general STS patient population.

The interim results of the Study illustrate an unexpected benefit. It is both unexpected and surprising that the combination of olaratumab and doxorubicin provides such a significant benefit to patients, especially as measured through progression free survival (PFS) and overall survival (OS) of patients.

First and foremost, the clinical data from the interim analysis of the Study are surprising because the data illustrate a 12-week improvement in PFS and, perhaps even more importantly, a 40-week improvement of OS, with a hazard ratio (HR) of 0.597 (90% confidence interval=0.415, 0.858) for PFS and a HR of 0.46 (90% confidence interval=0.288, 0.735) when olaratumab is combined with doxorubicin as compared to doxorubicin alone (the standard of care). Although the Study was not statistically powered for OS, a significant improvement over the current standard of care in terms of a lower HR and a larger improvement in median survival time are highly unexpected.

Second, based on the design of the Study alone, these improvements are unexpected because the Study was designed to allow detection of an improvement in PFS from 2 months (estimated from published clinical data in various types of patients with STS who were previously treated with one active agent and based on clinical assessment) to 3 months; in other words, per the Study design, a 4-week improvement is considered successful. The combination of olaratumab plus doxorubicin showed a 12-week improvement in PFS; the combination tripled the Study requirement under which the Study is considered successful. This illustrates an unexpected benefit over the anticipated Study outcome that is based on the standard of care, and the prior art when the Study was designed and initiated. When PFS and OS are compared to the currently available and recently investigated treatments referenced supra, the results are noteworthy and illustrate a clear unexpected benefit.

According to a first aspect of the present invention, there is provided a method of treating a patient having soft tissue sarcoma, comprising administering to the patient in need thereof olaratumab and doxorubicin. In a preferred aspect of the invention, the olaratumab is administered at a dose of about 15 mg/kg. In another aspect of the invention, the doxorubicin is administered at a dose of about 60 mg/m² or about 75 mg/m². In a preferred aspect of the invention, the doxorubicin is administered at a dose of about 75 mg/m². In yet another preferred aspect of the invention, the olaratumab is administered before the doxorubicin is administered. In a preferred aspect of the invention, the soft tissue sarcoma is leiomyosarcoma.

In another aspect of the present invention, a kit comprises olaratumab and doxorubicin, wherein the olaratumab and the doxorubicin are to be administered simultaneously, separately or sequentially.

Yet another aspect of the present invention is a kit comprising a pharmaceutical composition comprising olaratumab, with one or more pharmaceutically acceptable carriers, diluents, or excipients, and a pharmaceutical composition comprising doxorubicin, with one or more pharmaceutically acceptable carriers, diluents, or excipients, wherein the olaratumab and the doxorubicin are to be administered simultaneously, separately or sequentially.

Another aspect of the invention is a pharmaceutical composition comprising olaratumab with one or more pharmaceutically acceptable carriers, diluents, or excipients, in combination with a pharmaceutical composition of doxorubicin with one or more pharmaceutically acceptable carriers, diluents, or excipients, wherein the olaratumab and the doxorubicin are to be administered simultaneously, separately or sequentially for use in the treatment of soft tissue sarcoma.

Yet another aspect of the invention is use of olaratumab in the manufacture of a medicament for the treatment of soft tissue sarcoma, wherein the medicament is to be administered simultaneously, separately or sequentially with doxorubicin.

The invention also relates to a combination of olaratumab and doxorubicin for simultaneously, separately or sequentially use in the treatment of soft tissue sarcoma.

Olaratumab for simultaneous, separate or sequential use in combination with doxorubicin in the treatment of soft tissue sarcoma is another aspect of the invention.

In a preferred aspect of the invention relating to the pharmaceutical composition disclosed above, the use disclosed above, a combination disclosed above and/or olaratumab for a use disclosed above, the olaratumab is administered at a dose of about 15 mg/kg.

In another aspect of the invention relating to the pharmaceutical composition disclosed above, the use disclosed above, a combination disclosed above and/or olaratumab for a use disclosed above, the doxorubicin is administered at a dose of about 60 mg/m² or about 75 mg/m².

In a preferred aspect of the invention relating to the pharmaceutical composition disclosed above, the use disclosed above, a combination disclosed above and/or olaratumab for a use disclosed above, the doxorubicin is administered at a dose of about 75 mg/m².

In yet another preferred aspect of the invention relating to the pharmaceutical composition disclosed above, the use disclosed above, a combination disclosed above and/or olaratumab for a use disclosed above, the olaratumab is administered before the doxorubicin is administered.

In yet another preferred aspect of the invention relating to the pharmaceutical composition disclosed above, the use disclosed above, a combination disclosed above and/or olaratumab for a use disclosed above, the soft tissue sarcoma is leiomyosarcoma.

The present invention also contemplates the following non-limiting list of embodiments, which are further described elsewhere herein:

In reference to the various aspect disclosed above, soft tissue sarcoma is disease consisting of but not limited to: leiomyosarcoma, alveolar soft part sarcoma, chondroblastic osteosarcoma, chondrosarcoma, clear cell sarcoma, endometrial stromal sarcoma, epithelial sarcoma, epithelioid, extraskeletal myxoid chondrosarcoma, fibromyxoid sarcoma, fibrosarcoma, fibrosarcomatous transformation in a recurrent dermatofibrosarcoma, hemangiopericytoma, high grade undifferentiated sarcoma, liposarcoma, malignant fibrous histiocytoma, malignant glomus tumor, malignant peripheral nerve sheath tumor, malignant solitary fibrous tumor, malignant spindle cell sarcoma, malignant spindle cell tumor with rhabdoid features, myxofibrosarcoma, myxoid chondrosarcoma, myxoid liposarcoma, myxoid sarcoma, neurofibrosarcoma, osteosarcoma, pleomorphic sarcoma (including: high grade spindle and pleomorphic sarcoma of left thigh, poorly differentiated, round cell sarcoma, sarcoma, smooth muscle tumor, solitary fibrous tumor, spindle cell sarcoma, undifferentiated), rhabdomyosarcoma, synovial sarcoma, undifferentiated sarcoma, and undifferentiated uterine sarcoma. The soft tissue sarcoma may also be selected from the group consisting of leiomyosarcoma and other soft tissue sarcomas. The soft tissue sarcoma may be at an advanced stage.

According to a preferred embodiment of the present invention, there is provided a combination or a pharmaceutical composition comprising olaratumab and doxorubicin for separate, simultaneous or sequential use in therapy wherein the combination or pharmaceutical composition is administered parenterally.

According to a preferred embodiment of the present invention, there is provided a combination olaratumab and doxorubicin for separate, simultaneous or sequential use in therapy wherein the olaratumab is administered on a 21-day cycle on day one and day eight, wherein each dose of olaratumab falls within the range of about 10 mg/kg to about 18 mg/kg. Preferably, the dose is in the range of about 13.5 mg/kg to about 16.5 mg/kg and most preferably is about 15 mg/kg. Preferably, patients should be treated in cycles of 21 days until evidence of confirmed disease progression.

According to another preferred embodiment of the present invention, there is provided a combination of olaratumab and doxorubicin for separate, simultaneous or sequential use in therapy wherein the doxorubicin is administered on a 21-day cycle on day one and day eight, wherein each dose of doxorubicin falls within the range of about 60 mg/m² to about 75 mg/m². Preferably, the dose is about 60 mg/m² and most preferably is about 75 mg/m².

According to a further preferred embodiment of the present invention, there is provided a combination comprising olaratumab and doxorubicin for sequential use in therapy, wherein the doxorubicin is administered after the administration of the olaratumab.

According to another embodiment of the present invention, there is provided a combination comprising olaratumab and doxorubicin for sequential use in therapy, wherein the doxorubicin is administered one hour after the administration of the olaratumab.

The invention also provides for administering olaratumab at repeated intervals. Preferably, where olaratumab is administered at repeated intervals, the doxorubicin will be administered after the administration of olaratumab. In another embodiment, when the olaratumab is administered at repeated intervals, the doxorubicin will be administered one hour after the administration of the olaratumab.

The invention also provides for olaratumab for simultaneous, separate or sequential use in combination with doxorubicin in the treatment of soft tissue sarcoma, wherein the olaratumab is administered at a dose of about 15 mg/kg.

The invention also provides for olaratumab for simultaneous, separate or sequential use in combination with doxorubicin in the treatment of soft tissue sarcoma, wherein the olaratumab is administered at a dose of about 15 mg/kg and the olaratumab is administered before the doxorubicin is administered.

The invention also provides for olaratumab for simultaneous, separate or sequential use in combination with doxorubicin in the treatment of soft tissue sarcoma, wherein the olaratumab is administered at a dose of about 15 mg/kg and the doxorubicin is administered at a dose of about 60 mg/m² or about 75 mg/m².

The invention also provides for olaratumab for simultaneous, separate or sequential use in combination with doxorubicin in the treatment of soft tissue sarcoma, wherein the olaratumab is administered at a dose of about 15 mg/kg, the doxorubicin is administered at a dose of about 60 mg/m² or about 75 mg/m², and the olaratumab is administered before the doxorubicin is administered.

The invention also provides for olaratumab for simultaneous, separate or sequential use in combination with doxorubicin in the treatment of soft tissue sarcoma, wherein doxorubicin is administered at a dose of about 75 mg/m² and the olaratumab is administered before the doxorubicin is administered.

Preferably, the invention also provides for olaratumab for simultaneous, separate or sequential use in combination with doxorubicin in the treatment of soft tissue sarcoma, wherein the olaratumab is administered at a dose of about 15 mg/kg and the doxorubicin is administered at a dose of about 75 mg/m².

The invention also provides for olaratumab for simultaneous, separate or sequential use in combination with doxorubicin in the treatment of soft tissue sarcoma, wherein the olaratumab is administered at a dose of about 15 mg/kg, the doxorubicin is administered at a dose of about 75 mg/m², and the olaratumab is administered before the doxorubicin is administered.

The invention also provides for olaratumab for simultaneous, separate or sequential use in combination with doxorubicin in the treatment of soft tissue sarcoma, wherein the olaratumab and the doxorubicin are dosed on an a 21-day cycle, the olaratumab is administered at a dose of about 15 mg/kg, the doxorubicin is administered at a dose of about 75 mg/m², and the olaratumab is administered before the doxorubicin is administered.

The invention also provides for olaratumab for simultaneous, separate or sequential use in combination with doxorubicin in the treatment of soft tissue sarcoma, wherein the olaratumab is administered at a dose of about 15 mg/kg and wherein the soft tissue sarcoma is leiomyosarcoma.

The invention also provides for olaratumab for simultaneous, separate or sequential use in combination with doxorubicin in the treatment of soft tissue sarcoma, wherein the olaratumab is administered at a dose of about 15 mg/kg and the olaratumab is administered before the doxorubicin is administered and wherein the soft tissue sarcoma is leiomyosarcoma.

The invention also provides for olaratumab for simultaneous, separate or sequential use in combination with doxorubicin in the treatment of soft tissue sarcoma, wherein the olaratumab is administered at a dose of about 15 mg/kg and the doxorubicin is administered at a dose of about 60 mg/m² or about 75 mg/m² and wherein the soft tissue sarcoma is leiomyosarcoma.

The invention also provides for olaratumab for simultaneous, separate or sequential use in combination with doxorubicin in the treatment of soft tissue sarcoma, wherein the olaratumab is administered at a dose of about 15 mg/kg, the doxorubicin is administered at a dose of about 60 mg/m² or about 75 mg/m², and the olaratumab is administered before the doxorubicin is administered and wherein the soft tissue sarcoma is leiomyosarcoma.

The invention also provides for olaratumab for simultaneous, separate or sequential use in combination with doxorubicin in the treatment of soft tissue sarcoma, wherein doxorubicin is administered at a dose of about 75 mg/m² and the olaratumab is administered before the doxorubicin is administered and wherein the soft tissue sarcoma is leiomyosarcoma.

Preferably, the invention also provides for olaratumab for simultaneous, separate or sequential use in combination with doxorubicin in the treatment of soft tissue sarcoma, wherein the olaratumab is administered at a dose of about 15 mg/kg and the doxorubicin is administered at a dose of about 75 mg/m² and wherein the soft tissue sarcoma is leiomyosarcoma.

The invention also provides for olaratumab for simultaneous, separate or sequential use in combination with doxorubicin in the treatment of soft tissue sarcoma, wherein the olaratumab is administered at a dose of about 15 mg/kg, the doxorubicin is administered at a dose of about 75 mg/m², and the olaratumab is administered before the doxorubicin is administered and wherein the soft tissue sarcoma is leiomyosarcoma.

The invention also provides for olaratumab for simultaneous, separate or sequential use in combination with doxorubicin in the treatment of soft tissue sarcoma, wherein the olaratumab and the doxorubicin are dosed on an a 21-day cycle, the olaratumab is administered at a dose of about 15 mg/kg, the doxorubicin is administered at a dose of about 75 mg/m², and the olaratumab is administered before the doxorubicin is administered and wherein the soft tissue sarcoma is leiomyosarcoma.

The invention described above also provides for the combination of olaratumab and doxorubicin providing a median OS of approximately 98.9 weeks (at 90% Confidence Interval [CI]: 89.6, NE [wherein NE denotes that the limit was not reached]) (at 95% CI: 70.9, NE), or at least 70.9 weeks. The invention also provides for the combination of olaratumab and doxorubicin providing a median PFS of approximately 29.9 weeks (at 90% CI: 23.7, 36.0) (at 95% CI: 22.3, 36.7), or at least 22.3 weeks. The invention also provides for the combination of olaratumab and doxorubicin providing approximately a 40-week improvement in OS as compared to doxorubicin alone (the standard of care). The invention also provides for the combination of olaratumab and doxorubicin providing approximately a 12-week improvement in PFS of as compared to doxorubicin alone (the standard of care). The invention also provides for combinations of the aforementioned unexpected benefits.

The invention provides for olaratumab in various aspects disclosed herein. Olaratumab is an antibody specific for human PDGFRα and comprising the sequences disclosed in TABLE 1: (1) the 6 CDR amino acid sequences (CDRH1, CDRH2, CDRH3, CDRL1, CDRL2, CDRL3); (2) the heavy chain variable region (VH) and the light chain variable region (VL); (3) the a heavy chain and the light chain; or (4) two heavy chains and two light chains.

The term “soft tissue sarcoma” or “STS,” as used herein, is a malignancy that originates in soft or connective tissue like fat, muscle, nerves, fibrous tissues, blood vessels, or deep skin tissues; all of mesenchymal origin. More than 50 histological subtypes of STS have been identified. Subtypes included in the Study included: leiomyosarcoma and “other sarcomas.” Due to low population prevalence, non-leiomysarcoma soft tissue sarcoma subtypes were represented in the Study collectively as “other sarcoma” for statistical purposes as discussed infra. As referred herein, “soft tissue sarcoma” or “STS” is defined as: alveolar soft part sarcoma, chondroblastic osteosarcoma, chondrosarcoma, clear cell sarcoma, endometrial stromal sarcoma, epithelial sarcoma, epithelioid, extraskeletal myxoid chondrosarcoma, fibromyxoid sarcoma, fibrosarcoma, fibrosarcomatous transformation in a recurrent dermatofibrosarcoma, hemangiopericytoma, high grade undifferentiated sarcoma, leiomyosarcoma, liposarcoma, malignant fibrous histiocytoma, malignant glomus tumor, malignant peripheral nerve sheath tumor, malignant solitary fibrous tumor, malignant spindle cell sarcoma, malignant spindle cell tumor with rhabdoid features, myxofibrosarcoma, myxoid chondrosarcoma, myxoid liposarcoma, myxoid sarcoma, neurofibrosarcoma, osteosarcoma, pleomorphic sarcoma (including: high grade spindle and pleomorphic sarcoma, of left thigh, poorly differentiated, round cell sarcoma, sarcoma, smooth muscle tumor, solitary fibrous tumor, spindle cell sarcoma, undifferentiated), rhabdomyosarcoma, synovial sarcoma, undifferentiated sarcoma, and undifferentiated uterine sarcoma. Due to the nature of the disease state and origin of the tumor, a patient may be diagnosed as one or more of the exemplary subtypes noted above; however, diagnosis may not be limited to the subtypes above.

Treatment options for STS, including subtypes exemplified in the Study, continue to be limited. Despite continued efforts, increased survival benefits have been modest. Even combinations with doxorubicin have shown little or no clinical benefit. There is a high clinical unmet need for new treatment options for patients with STS that provide a survival benefit; the present invention provides such.

As used herein, the term “advanced” STS refers to any of the following criteria including but not limited to: (1) unresectable, (2) metastatic, (3) histologically or cytologically confirmed documented disease progression, or (4) not amenable to treatment with surgery or radiotherapy.

The terms “platelet-derived growth factor receptor alpha,” “platelet-derived growth factor receptor α,” “PDGFR alpha,” “PDGFRα,” “PDGF alpha receptor,” and “PDGFα receptor” are used interchangeably herein, unless otherwise indicated, and are intended to refer to the human type III receptor tyrosine kinase, as well as functionally active, mutated forms thereof, that bind human platelet-derived growth factor. Specific examples of PDGFRα include, e.g., a human polypeptide encoded by the nucleotide sequence provided in GenBank® accession no. NM_006206.4, or the human protein encoded by the polypeptide sequence provided in GenBank® accession no. NP_006197.1.

PDGFRα is a receptor tyrosine kinase that can be activated by platelet-derived growth factor (PDGF)-AA, -AB, -BB, and -CC. These growth factors are dimeric molecules composed of disulfide-linked polypeptide chains that bind to two receptors simultaneously and induce receptor dimerization, autophosphorylation, and downstream intracellular signaling. PDGFRα is expressed in many mesenchymal structures; thus, PDGFRα plays a critical role during early and later stages of development.

As used herein, the term “olaratumab”—also known as IMC-3G3, CAS registry number 1024603-93-7—refers to an anti-PDGFRα antibody comprising: two heavy chains, each of whose amino acid sequence is that given in SEQ ID NO: 9, and two light chains, each of whose amino acid sequence is that given in SEQ ID NO: 10. U.S. Pat. Nos. 8,128,929 and 8,574,578.

Olaratumab is a recombinant human monoclonal antibody of the IgGᵢ isotype that specifically targets human PDGFRα. The antibody possesses high-affinity binding for PDGFRα and blocks platelet-derived growth factor-AA (PDGF-AA), -BB, and -CC ligands from binding to the receptor. As a result, olaratumab inhibits ligand-induced receptor autophosphorylation and phosphorylation of the downstream signaling molecules protein kinase B (Akt) and mitogen-activated protein kinase (MAPK). Olaratumab inhibits the proliferation and growth of a variety of human tumor cell lines.

As used herein, the term “antibody” includes immunoglobulin molecules comprising four polypeptide chains, two heavy (H) chains and two light (L) chains inter-connected by disulfide bonds. Individual chains can fold into domains having similar sizes (110-125 amino acids) and structures, but different functions. Antibody may be abbreviated herein as “Ab.”

The light chain can comprise one variable domain (VL) and/or one constant domain (abbreviated herein as CL). The light chains of human antibodies (immunoglobulins) are either kappa (K) light chains or lambda (λ) light chains. The expression VL, as used herein, is intended to include both the variable regions from kappa-type light chains (VK) and from lambda-type light chains (Vλ). The heavy chain can also comprise one variable domain (VH) and/or, depending on the class or isotype of antibody, three or four constant domains (CH1, CH2, CH3 and CH4) (abbreviated herein collectively as CH). In humans, the isotypes are IgA, IgD, IgE, IgG, and IgM, with IgA and IgG further subdivided into subclasses or subtypes (IgA₁₋₂ and IgG₁₋₄). The present invention includes antibodies of any of the aforementioned classes or subclasses. Human IgG₁ is the preferred isotype for the antibodies of the present invention.

Three regions, called hypervariable or complementarity-determining regions (hereinafter “CDRs”), are found in each of VL and VH, which are supported by less variable regions called frameworks (herein as “FR”). Amino acids are assigned to a particular CDR region or domain in accordance with various conventions including, but not limited to: Kabat (Kabat, et al., Sequences of Proteins of Immunological Interest, Fifth Edition, U.S. Department of Health and Human Services, NIH Publication No. 91-3242 (1991)), Chothia (Chothia, et al., J Mol Biol. 1987; 196: 901-917. Chothia, et al., Nature. 1989; 342: 877-883), and/or Oxford Molecular's AbM antibody modelling software (http://www.bioinf.org.uk/abs/). Each VH and VL is composed of three CDRs and four FRs, arranged from amino-terminus to carboxy-terminus in the following order: FR1-CDR1-FR2-CDR2-FR3-CDR3-FR4. The portion of an antibody consisting of VL and VH domains is designated Fv (Fragment variable) and constitutes the antigen-binding site.

The term “isolated” refers to an antibody, protein, peptide or nucleic acid that is free or substantially free from other macromolecular species found in a cellular environment. “Substantially free,” as used herein means the protein peptide or nucleic acid of interest comprises more than 80% (on a molar basis) of the macromolecular species present, preferably more than 90% and more preferably more than 95%. Examples of “isolated” antibodies include an antibody that has been affinity purified, an antibody that has been made by a hybridoma or other cell line in vitro, and a human antibody derived from a transgenic mouse.

The term “monoclonal,” as used herein, refers to an antibody obtained from a population of substantially homogeneous antibodies, e.g., the individual antibodies comprising the population are substantially identical except for possible naturally occurring mutations or minor post-translational variations that may be present. Monoclonal antibodies are highly specific, being directed against a single antigenic site (also known as determinant or epitope). Furthermore, in contrast to conventional (polyclonal) antibody preparations that typically include different antibodies directed against different determinants, each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. Monoclonal antibody may be abbreviated herein as “mAb.”

The term “human antibody,” as used herein, includes antibodies having variable and constant regions corresponding to human germline immunoglobulin sequences (as described in Kabat et al., supra). The human antibodies of the invention may include amino acid residues not encoded by human germline immunoglobulin sequences (e.g., mutations introduced by random or site-specific mutagenesis in vitro or by somatic mutation in vivo), for example in the CDRs. The human antibody can have at least one position replaced with an amino acid residue, e.g., an activity enhancing amino acid residue which is not encoded by the human germline immunoglobulin sequence. However, the term “human antibody,” as used herein, is not intended to include antibodies in which CDR sequences derived from the germline of another mammalian species, such as a mouse, have been grafted onto human framework sequences. Methods of producing a “human antibody,” as used herein are not intended to include antibodies produced in a human being.

The phrase “recombinant human antibody” includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal that is transgenic for human immunoglobulin genes, or antibodies prepared, expressed, created or isolated by any other means that involves splicing of human immunoglobulin gene sequences to other DNA sequences. Such recombinant human antibodies have variable and constant regions derived from human germline immunoglobulin sequences.

Thus, antibodies of the invention include, but are not limited to, isolated antibodies, human antibodies, humanized antibodies, recombinant human antibodies, monoclonal antibodies, digestion fragments, specified portions and variants thereof, including antibody mimetics or comprising portions of antibodies that mimic the structure and/or function of an antibody or specified fragment or portion thereof; each containing at least one CDR.

Specificity of antibodies or fragments thereof can be determined based on affinity. Affinity, represented by the equilibrium constant for the dissociation of an antigen with an antibody (K_{D}), measures the binding strength between an antigenic determinant and an antibody-binding site. Affinity can be measured for example by surface plasmon resonance.

The antibodies of the invention bind to an epitope of PDGFRα located on the extracellular domain segments (hereinafter referred simply to as “domains” or “ECD”). The term “epitope” as used herein refers to discrete, three-dimensional sites on an antigen that are recognized by the antibodies of the invention.

In addition to the antibodies specifically described herein, other “substantially homologous” modified antibodies can be readily designed and manufactured utilizing various recombinant DNA techniques well known to those skilled in the art. For example, the framework regions can vary from the native sequences at the primary structure level by several amino acid substitutions, terminal and intermediate additions and deletions, and the like. Moreover, a variety of different human framework regions may be used singularly or in combination as a basis for the humanized immunoglobulins of the present invention. In general, modifications of the genes may be readily accomplished by a variety of well-known techniques, such as site-directed mutagenesis.

The present invention includes nucleic acid sequences that encode an anti-PDGFRα antibody heavy chain, comprising any one of the VH regions or a portion thereof, or any one of the VH CDRs, including any variants thereof, as disclosed herein. The invention also includes nucleic acid molecules that encode an anti-PDGFRα antibody light chain comprising any one of the VL regions, or a portion thereof or any one of the VL CDRs, including any variants thereof as disclosed herein. The invention also includes the nucleic acid sequences of olaratumab, SEQ ID NOs 11 and 12, for heavy chain and light chain, respectively. The antibodies of the invention include antibodies comprising the same CDR regions of olaratumab, and/or the same light chain variable region and/or heavy chain variable region of olaratumab.

The antibodies of the present invention may be produced by methods known in the art. These methods include the use of transgenic animal, phage display and the immunological method described by Kohler and Milstein, Nature 256: 495-497 (1975); Laboratory Techniques in Biochemistry and Molecular Biology, Volume 13 (Burdon et al. eds., Elsevier Science Publishers, Amsterdam) in Monoclonal Antibody Technology, The Production and Characterization of Rodent and Human Hybridomas (Campbell ed., 1984); as well as by the recombinant DNA method described by Huse et al., Science 246: 1275-1281 (1989).

It is understood that amino acid residues that are primary determinants of binding of single domain antibodies can be within Kabat, Chothia, AbM, or a combination thereof defined CDRs, but may include other residues as well, such as residues that would otherwise be buried in the VH-VL interface of a VH-VL heterodimer.

Preferred host cells for transformation of vectors and expression of the antibodies of the present invention are mammalian cells, e.g., NSO cells, 293, SP20, CHO cells, and other cell lines of lymphoid origin such as lymphoma, myeloma, or hybridoma cells. Other eukaryotic hosts such as yeasts can be alternatively used.

The antibodies of the present invention may be isolated or purified by any method known in the art, including precipitation by ammonium sulfate or sodium sulfate followed by dialysis against saline, ion exchange chromatography, affinity or immuno-affinity chromatography, as well as gel filtration or zone electrophoresis. A preferred method of purification for the antibodies of the current invention is Protein-A affinity chromatography.

As used herein, “about” means ±5%.

As used herein, “dexrazoxane” or dexrazoxane hydrochloride is a cardioprotective agent. It is commonly used to protect the heart against the cardiotoxic side effects of anthracyclines, such as daunorubicin or doxorubicin.

As used herein, the terms “treating,” “treat,” or “treatment” refers to restraining, slowing, lessening, reducing, or reversing the progression or severity of an existing symptom, disorder, condition, or disease or ameliorating clinical symptoms of a condition. Beneficial or desired clinical results include, but are not limited to, alleviation of symptoms, diminishment of the extent of a disease or disorder, stabilization of a disease or disorder (i.e., where the disease or disorder does not worsen), delay or slowing of the progression of a disease or disorder, amelioration or palliation of the disease or disorder, and remission (whether partial or total) of the disease or disorder, whether detectable or undetectable. Treatment can also mean prolonging survival as compared to expected survival if not receiving treatment. Those in need of treatment include those already with the disease. In one embodiment, the present invention can be used as a medicament.

As used herein, the terms “cancer” and “cancerous” refer to or describe the physiological condition in mammals that is typically characterized by unregulated cell growth. Included in this definition are benign and malignant cancers.

Although human antibodies of the invention are particularly useful for administration to humans, they can be administered to other mammals as well. Accordingly, as used herein, the term “patient” refers to a mammal, preferably a human. The term mammal as used herein is intended to include, but is not limited to, humans, laboratory animals, domestic pets and farm animals.

A therapeutically effective amount is also one in which any toxic or detrimental effects of the antibody or antibody portion are outweighed by the therapeutically beneficial effects.

In the methods of the present invention, a therapeutically effective amount of an antibody of the invention is administered to a mammal or patient in need thereof. Additionally, the pharmaceutical compositions of the invention may include a therapeutically effective amount of an anti-PDGFRα antibody of the invention or a therapeutically effective amount of doxorubicin.

A “therapeutically effective amount,” “effective amount” or “effective dose” as used herein, refers to an amount effective, at dosages and for periods of time necessary, to achieve the desired therapeutic result. An effective amount can be readily determined by the attending diagnostician, as one skilled in the art, by the use of known techniques and by observing results obtained under analogous circumstances. In determining the effective amount for a patient, a number of factors are considered by the attending diagnostician, including, but not limited to: the species of patient; its size, age, and general health; the specific disease or disorder involved; the target site; the degree of or involvement or the severity of the disease or disorder; the response of the individual patient; the particular compound administered; the mode of administration; the bioavailability characteristics of the preparation administered; the dose regimen selected; the use of concomitant medication; other medications administered; and other relevant circumstances. A therapeutically effective amount is also one in which any toxic or detrimental effects of the antibody or antibody portion are outweighed by the therapeutically beneficial effects.

Generally, dosage regimens may be adjusted to provide the optimum desired response (e.g., a therapeutic response). Treatment dosages may be titrated using routine methods known to those of skill in the art to optimize safety and efficacy. Dosing schedules will typically range from a single bolus dosage or continuous infusion to multiple administrations per day (e.g., every 4-6 hours), or as indicated by the treating physician and the patient's condition. An exemplary, non-limiting range for a therapeutically effective amount of an antibody of the invention is 0.1-50 mg/kg, more preferably 3-35 mg/kg, and more preferably 5-20 mg/kg. Dosing amounts and frequencies of the antibody will be determined by the physicians treating the patient and may include doses from less than 1 mg/kg to over 100 mg/kg given daily, three times per week, weekly, once every two weeks, or less often. It should be noted, however, that the present invention is not limited to any particular dose.

Olaratumab is generally effective over a wide dosage range in the combination of the present invention. For example, dosages normally are given on a 21-day cycle normally on days one and eight and each dose falls within the range of about 10 mg/kg to about 18 mg/kg, preferably about 13.5 mg/kg to about 16.5 mg/kg, and most preferably about 15 mg/kg. Preferably, patients should be treated in cycles of 21 days until evidence of confirmed disease progression.

Doxorubicin is generally effective over a wide dosage range in the combination of the present invention; however, the standard dose for doxorubicin in STS is 60 mg/m² or 75 mg/m². Accordingly, for example, dosages per 21-day cycle normally are about 60 mg/m² or 75 mg/m², preferably about 75 mg/m². With doxorubicin, exceeding eight 21-day cycles is generally not recommended due to the unacceptably high rate of cardiac insufficiency starting at a cumulative dose of 600 mg/m².

In some instances, dosage levels below the lower limit of the aforesaid ranges for olaratumab and doxorubicin may be more than adequate; while in other cases, smaller or still larger doses may be employed with acceptable side effects; therefore, the above dosage range is not intended to limit the scope of the invention in any way. When olaratumab and doxorubicin are given in combination, they are administered in the same ranges as described above.

As used herein, the terms “effective response” of a patient or a patient's “responsiveness” to treatment with of the agents, or “therapeutic effect” refers to the clinical or therapeutic benefit(s) imparted to a patient upon administration. As used herein, an “unexpected therapeutic effect” of the treatment of the invention is the ability to produce marked anti-cancer effects in a patient without causing significant toxicities or adverse effects, so that the patient benefits from the treatment overall. The efficacy, i.e., therapeutic effect(s), of the treatment of the invention can be measured by various endpoints commonly used in evaluating cancer treatments, include any one or more including, but not limited to: extending survival (including OS and PFS); resulting in an objective response (including a CR or a PR); tumor regression, tumor weight or size shrinkage, longer time to disease progression, increased duration of survival, longer PFS, improved OS rate, increased duration of response, and improved quality of life and/or improving signs or symptoms of cancer, etc. Because the invention relates to the use of a combination of unique anti-tumor agents, novel approaches to determine efficacy, i.e., therapeutic effect(s), of any particular combination therapy of the present invention can be optionally employed, including, for example, measurement of plasma or urinary markers of angiogenesis and measurement of response through radiological imaging.

As used herein, the term “disease progression” or “progressive disease” (PD), used interchangeably herein, refers to least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. The appearance of one or more new lesions is also considered progression.

As used herein, the term “partial response,” (PR) refers to least a 30% decrease in the sum of diameters of target lesions, taking as reference the baseline sum diameters.

As used herein, the term, “complete response” (CR) refers to the disappearance of all target lesions. Any pathological lymph nodes (whether target or non-target) must have reduction in short axis to <10 mm.

As used herein, the term “stable disease” (SD) refers to a tumor diameter that does not shrink sufficiently to qualify for PR or sufficient increase to qualify for PD, taking as reference the smallest sum diameters while on Study.

As used herein, the term “objective response” (OR) refers to a measurable response, including CR or PR.

As used herein, the term, “overall survival” (OS) refers to the patient remaining alive for a defined period of time, such as 1 year, 5 years, etc. from the time of diagnosis or treatment. In a preferred aspect of the invention, for the Study, overall survival is defined as the time from the date of randomization in the Study to the date of death from any cause; if the patient is alive at the end of the follow-up period or is lost to follow-up, OS will be censored on the last date the patient is known to be alive.

As used herein, the term, “progression-free survival” (PFS) refers to the patient remaining alive without the cancer progressing or getting worse. In a preferred aspect of the invention, PFS is defined as the time from randomization in the Study until the first radiographic documentation of objective progression as defined by RECIST (Version 1.1), or death from any cause. Patients who die without a reported prior progression will be considered to have progressed on the day of their death. Patients who did not progress or are lost to follow-up will be censored at the day of their last radiographic tumor assessment.

As used herein, the term “extending survival” or “prolonged survival” which are used interchangeably herein, refers to increasing OS or PFS in a treated patient relative to i) an untreated patient, ii) a patient treated with less than all of the anti-tumor agents in a particular combination therapy, or iii) a control treatment protocol. Survival is monitored for at least about one month, at least about two months, at least about four months, at least about six months, at least about nine months, or at least about 1 year, or at least about 2 years, or at least about 3 years, or at least about 4 years, or at least about 5 years, or at least about 10 years, etc., following the initiation of treatment or following the initial diagnosis of cancer.

Anti-PDGFRα antibodies can be administered in combination with one or more other anti-cancer treatments including but not limited to an anti-angiogenesis agent, a chemotherapeutic agent, and an anti-neoplastic agent. Any suitable anti-cancer agent can be used, such as a chemotherapeutic agent, radiation, antibody or combinations thereof. Anti-cancer agents include but are not limited to anti-neoplastic agents, antibodies, adjuvants, and prodrugs. Anti-PDGFRα antibodies of the invention can be administered with antibodies and/or small molecules that inhibit and/or modulate other cell surface receptors involved in tumor growth or angiogenesis or extracellular matrix proteins/factors or epithelial/mesenchymal transitions. It can also be administered in combination with one or more suitable adjuvants, such as, for example, cytokines or other immune stimulators, such as, but not limited to, chemokine, tumor-associated antigens, and peptides.

In the present invention, any suitable method or route can be used to administer anti-PDGFRα antibodies of the invention, and optionally, to co-administer anti-neoplastic agents and/or antagonists of other receptors. In a combination therapy of the present invention, the anti-PDGFRα antibody can be administered before, during, substantially simultaneous with, or after commencing therapy with another agent. Routes of administration include, for example, oral, intravenous, intraperitoneal, subcutaneous, or intramuscular administration; intravenous administration is the preferred route. It should be emphasized, however, that the present invention is not limited to any particular method or route of administration.

The anti-PDGFRα antibodies of the invention, where used in a mammal for the purpose of treatment, are preferably formulated as pharmaceutical compositions. Such pharmaceutical compositions and processes for preparing the same are well known in the art. See, e.g. Remington: The Science and Practice of Pharmacy (Gennaro A., et al., eds., 19th ed., Mack Publishing Co., 1995).

Olaratumab and doxorubicin are preferably formulated as pharmaceutical compositions administered by any route that makes the compound bioavailable. The route of administration may be varied in any way, limited by the physical properties of the drugs and the convenience of the patient and the caregiver. Preferably, olaratumab and doxorubicin compositions are for parenteral administration, such as intravenous (i.v.) administration. Such pharmaceutical compositions and processes for preparing same are well known in the art. (See e.g., id.). The route of administration may be varied in any way, limited by the physical properties of the drugs and the convenience of the patient and the caregiver.

Olaratumab and doxorubicin may be administered simultaneously, separately, or sequentially. As used herein, the phrase “in combination with” refers to the administration of olaratumab and doxorubicin simultaneously, sequentially in any order, or any combination thereof. The two compounds may be administered in separate pharmaceutical compositions. Olaratumab can be administered prior to, at the same time as, or subsequent to administration of doxorubicin or in some combination thereof. In a preferred aspect, the doxorubicin will be administered after the administration of olaratumab. In another aspect, the doxorubicin will be administered one hour after the administration of olaratumab. Where olaratumab is administered at repeated intervals (e.g. during a standard course of treatment), doxorubicin can be administered prior to, at the same time as, or subsequent to each administration of olaratumab or some combination thereof, or at different intervals in relation to therapy with olaratumab or in a single or series of dose(s) prior to, at any time during, or subsequent to the course of treatment with olaratumab. In a preferred aspect where olaratumab is administered at repeated intervals, the doxorubicin will be administered after the administration of olaratumab. In another aspect where olaratumab is administered at repeated intervals, the doxorubicin will be administered one hour after the administration of olaratumab.

As used herein, the term “kit” refers to a package comprising at least two separate containers, wherein a first container contains olaratumab and a second container contains doxorubicin. A “kit” may also include instructions to administer all or a portion of the contents of these first and second containers to a cancer patient, preferably a STS patient. Optionally, these kits also include a third container containing another anti-neoplastic agent.

The following examples illustrate the unexpected benefit of the present combinations.

### EXAMPLES AND ASSAYS

The following examples and assays further illustrate the invention, but should not be construed to limit the scope of the invention in any way. Detailed descriptions of conventional methods, such as those employed in the construction of vectors and plasmids, the insertion of genes encoding polypeptides into such vectors and plasmids, the introduction of plasmids into host cells, and the expression and determination thereof of genes and gene products can be obtained from numerous publications, including Sambrook, J. et al., Molecular Cloning: A Laboratory Manual, 2nd ed., Cold Spring Harbor Laboratory Press (1989) and Coligan, J. et al. Current Protocols in Immunology, Wiley & Sons, Incorporated (2007).

### Engineering, Expression and Purification of Human Anti-PDGFRα Antibodies

For each antibody (U.S. Pat. Nos. 8,128,929 and 8,574,578), engineer a suitable heavy chain nucleotide sequence, for example SEQ ID NO. 11 for olaratumab, into a suitable expression plasmid and engineer a suitable light chain nucleotide sequence, for example SEQ ID NO. 12 for olaratumab, into a suitable expression plasmid by a suitable method such as PCR cloning. To establish a stable cell line, transfect in a suitable host cell line, such as NSO or CHO cells, with linearized heavy and light chain plasmids and culture in suitable media such as glutamine free Dulbecco's Modified Eagle Medium with dialyzed fetal calf serum and glutamine synthetase supplement. Screen clones for antibody expression by an enzyme-linked immunosorbent assay (ELISA) and select the highest producer for culture in spinner flasks. Purify antibodies by a suitable method such as protein-A affinity chromatography.

Table 1 provides the amino acid sequences and corresponding SEQ ID NOs. of the antibody of the present invention. All CDR sequences are determined using the Kabat convention. Polynucleic acid sequences that encode the amino acid sequences disclosed below are also included within the scope of the present invention.

TABLE 1
Amino Acid Sequence of Olaratumab
Heavy and Light Chain Variable Region CDRs

| | | SEQ | | SEQ |
|---|---|---|---|---|
| | | ID | | ID |
| | Heavy Chain | NO. | Light Chain | NO. |
| CDR1 | SSSYYWG | 1 | RASQSVSSYLA | 4 |
| CDR2 | SFFYTGSTYYNPSLRS | 2 | DASNRAT | 5 |
| CDR3 | QSTYYYGSGNYYGWFDR | 3 | QQRSNWPPA | 6 |
| Variable | QLQLQESGPGLVKPSETLSLT | 7 | EIVLTQSPATLSLSPGERA | 8 |
| region | CTVSGGSINSSSYYWGWLRQ | | TLSCRASQSVSSYLAWYQ | |
| | SPGKGLEWIGSFFYTGSTYY | | QKPGQAPRLLIYDASNRA | |
| | NPSLRSRLTISVDTSKNQFSL | | TGIPARFSGSGSGTDFTLTI | |
| | MLSSVTAADTAVYYCARQS | | SSLEPEDFAVYYCQQRSN | |
| | TYYYGSGNYYGWFDRWDQ | | WPPAFGQGTKVEIK | |
| | GTLVTVSS | | | |
| Full | MGWSCIILFLVATATGVHSQ | 9 | MGWSCIILFLVATATGVH | 10 |
| Length | LQLQESGPGLVKPSETLSLTC | | SEIVLTQSPATLSLSPGERA | |
| | TVSGGSINSSSYYWGWLRQS | | TLSCRASQSVSSYLAWYQ | |
| | PGKGLEWIGSFFYTGSTYYN | | QKPGQAPRLLIYDASNRA | |
| | PSLRSRLTISVDTSKNQFSLM | | TGIPARFSGSGSGTDFTLTI | |
| | LSSVTAADTAVYYCARQST | | SSLEPEDFAVYYCQQRSN | |
| | YYYGSGNYYGWFDRWDQG | | WPPAFGQGTKVEIKRTVA | |
| | TLVTVSSASTKGPSVFPLAPS | | APSVFIFPPSDEQLKSGTA | |
| | SKSTSGGTAALGCLVKDYFP | | SVVCLLNNFYPREAKVQ | |
| | EPVTVSWNSGALTSGVHTFP | | WKVDNALQSGNSQESVT | |
| | AVLQSSGLYSLSSVVTVPSSS | | EQDSKDSTYSLSSTLTLSK | |
| | LGTQTYICNVNHKPSNTKVD | | ADYEKHKVYACEVTHQG | |
| | KRVEPKSCDKTHTCPPCPAP | | LSSPVTKSFNRGEC | |
| | ELLGGPSVFLFPPKPKDTLMI | | | |
| | SRTPEVTCVVVDVSHEDPEV | | | |
| | KFNWYVDGVEVHNAKTKPR | | | |
| | EEQYNSTYRVVSVLTVLHQ | | | |
| | DWLNGKEYKCKVSNKALPA | | | |
| | PIEKTISKAKGQPREPQVYTL | | | |
| | PPSREEMTKNQVSLTCLVKG | | | |
| | FYPSDIAVEWESNGQPENNY | | | |
| | KTTPPVLDSDGSFFLYSKLTV | | | |
| | DKSRWQQGNVFSCSVMHEA | | | |
| | LHNHYTQKSLSLSPGK | | | |

### A Randomized Phase 2 Study Evaluating the Efficacy of Doxorubicin With or Without Olaratumab in the Treatment of Soft Tissue Sarcoma (“The Study”)

### Study Design:

The Study is an open-label, multicenter, Phase 2 trial in which patients with advanced STS not amenable to treatment with surgery or radiotherapy are treated with olaratumab in combination with doxorubicin or doxorubicin alone.

Enrolled patients who meet all eligibility criteria are randomized 1:1 to one of two treatment arms, Arm A or Arm B. Arm A patients receive olaratumab in combination with doxorubicin (hereinafter the “Combination Arm”) and Arm B patients receive doxorubicin alone (hereinafter the “Doxorubicin Arm”). Randomization of this Study is performed according to a dynamic randomization algorithm using 4 pre-defined risk factors: PDGFRα expression (positive vs. negative), number of previous lines of treatment (0 vs. 1+lines), histological tumor type (i.e., leiomyosarcoma vs. synovial sarcoma vs. other tumor type), and ECOG performance status (0-1 vs. 2). This dynamic randomization procedure is used to minimize imbalance between treatment groups. To determine tumor type and PDGFRα expression in patients, a tumor sample from either a biopsy or previously archived tumor tissue sample is sufficient. Biopsy is required to be performed within 21 days prior to randomization; archived tissue sample must also be available within this timeframe.

Combination Arm: 64 patients randomized in the Combination Arm receive 15 mg/kg of olaratumab on Days 1 and 8 of each 21-day treatment cycle, in combination with 75 mg/m² doxorubicin administered on Day 1 of each cycle. (Note: if premedication is required prior to the first doxorubicin infusion, this is to be done after the completion of olaratumab infusion, not before the olaratumab infusion. This premedication may be administered within the hour that follows completion of the olaratumab infusion.) The actual (not planned) administration of each doxorubicin infusion defines Day 1 of each 21-day treatment cycle. The combination treatment just described is to continue for up to 8 cycles at the investigator's discretion. During Cycles 5 through 8, patients being treated with doxorubicin may also be administered 750 mg/m² dexrazoxane on Day 1 of each cycle, (at the investigator's discretion) prior to doxorubicin on Day 1 of each 21-day cycle. If the dose of doxorubicin is reduced at any point in the Study, the dose of dexrazoxane is to be reduced accordingly, maintaining a dosage ratio of 10:1. For example, if doxorubicin is reduced to 60 mg/m², then dexrazoxane is to be reduced to 600 mg/m². Doxorubicin may be administered within the hour that follows completion of the olaratumab infusion. In the absence of PD or other withdrawal criteria, patients in the Combination Arm who have completed eight cycles of therapy are to receive subsequent olaratumab monotherapy, administered on Days 1 and 8 of each 21-day cycle. Furthermore, if doxorubicin is discontinued (e.g., for reasons of toxicity) prior to end of cycle 8 and in the absence of withdrawal criteria, patients may continue to receive olaratumab monotherapy. For patients who discontinue olaratumab during the first 8 cycles of treatment, doxorubicin may be continued (for up to 8 cycles), provided no withdrawal criteria are met.

Doxorubicin Arm: 65 patients randomized in the Doxorubicin Arm receive 75 mg/m² doxorubicin on Day 1 of each cycle, for up to 8 cycles at the investigator's discretion. The actual (not planned) administration of each doxorubicin infusion defines Day 1 of each 21-day treatment cycle. During cycles 5 through 8, 750 mg/m² dexrazoxane may also be administered on Day 1 of each cycle, (at the investigator's discretion) prior to doxorubicin on Day 1 of each 21-day cycle. If the dose of doxorubicin is reduced at any point in the Study, the dose of dexrazoxane is to be reduced accordingly, maintaining a dosage ratio of 10:1. For example, if doxorubicin is reduced to 60 mg/m², then dexrazoxane is to be reduced to 600 mg/m². Doxorubicin Arm patients who develop PD during the initial 8 cycles may cross over to receive olaratumab monotherapy at the same dosing regimen and duration similar to those patients from the Combination Arm who continue on olaratumab monotherapy. Furthermore, patients in the Doxorubicin Arm who are discontinuing Study therapy due to therapy-related toxicity or complete doxorubicin treatment and experience SD or better may stay on Study schedule and assessments until PD is documented and then receive olaratumab monotherapy. In the case that a Doxorubicin Arm patient is to receive olaratumab monotherapy, PD must meet the protocol criteria.

Patients are assessed for tumor response every 6 weeks. Treatment is to continue until PD, development of unacceptable toxicity, noncompliance or withdrawal of consent by the patient, or investigator decision. Approximately 130 patients (65 in each treatment arm) were enrolled in the Phase 2 stage of this Study.

### Analysis of Efficacy Data

As per the pre-defined Study criteria, the Study is considered complete when the final analysis for OS is performed (which includes survival data for up to 24 months [2 years] after the last patient has been treated with his/her first dose of Study medication). After this point, any patients who are deriving benefit on Study treatment may continue to receive Study treatment after Study completion, and this treatment extension is to be decided on an individual basis.

The pre-defined end of trial is defined as when two years after the last patient has been treated with his/her first dose of Study medication, and the last patient has discontinued Study treatment and completed the 30-day safety follow-up visit or all olaratumab-related adverse events (AEs) have been followed until resolved, stabilized, returned to baseline, or deemed irreversible, whichever is the latest.

For Phase 2 data, the interim analysis looking at the efficacy data is scheduled after at least 80 PFS events are observed. The interim analysis mainly assesses the efficacy in terms of PFS. Other selected secondary variables such as OS and OR may also be evaluated.

### Analysis of Efficacy Data of Phase 2 Patients

For patients crossing over to olaratumab monotherapy after PD on the Doxorubicin Arm, all efficacy endpoints except OS are summarized separately for the post-cross-over period.

### Primary Efficacy Endpoint

PFS is defined as the time from randomization until the first radiographic documentation of objective progression, or death from any cause. Patients who die without a reported prior progression are considered to have progressed on the day of their death. Patients who did not progress or are lost to follow-up are censored at the day of their last radiographic tumor assessment. If no baseline or post-baseline radiologic assessment is available, the patient is censored at the date of randomization. If death or PD occurs after two or more consecutive missing radiographic visits, censoring occurs at the date of the last radiographic visit prior to the missed visits. The use of a new anticancer therapy prior to the occurrence of PD results in censoring at the date of last radiographic assessment prior to initiation of new therapy.

As a sensitivity analysis, the actual reported date of progression and death is used to define PFS regardless of missing visits, early discontinuation, or start of new therapies to avoid informative censoring. Additionally, symptomatic deterioration may also be added as a progressive event for another sensitivity analysis.

The Kaplan-Meier method is used to estimate the median PFS time, together with a 90% confidence interval (hereinafter “CI”). The 3-month PFS is also estimated. Comparison between arms is performed using the Log-rank test, and the hazard ratios (HR) are estimated by a Cox proportional hazards regression model. Only when there are a sufficient number of patients in each stratum, the stratified analysis may be performed. Otherwise, the stratification factors may be treated as covariates in the Cox model to estimate the HR and 90% confidence limit.

Secondary Efficacy Endpoints

Secondary efficacy endpoints include OS, Objective Response Rate (“ORR”), and the association between tumor PDGFRα expression and clinical outcomes, including PFS, ORR, etc. The analysis of the secondary endpoints may be adjusted for the stratification factors.

OS is defined as the time from the date of randomization to the date of death from any cause. If the patient is alive at the end of the follow-up period or is lost to follow-up, OS is censored on the last date the patient is known to be alive. OS is evaluated by the Kaplan-Meier method and a 90% CI is provided for the median OS. The HR and 90% confidence limit for OS are estimated from the Cox regression model, taking the stratification factors as covariates. As a sensitivity analysis, patients in the Doxorubicin Arm are censored at the date of cross-over, since the OS endpoint is confounded by cross-over.

The ORR is equal to the proportion of patients achieving a best overall response of PR or CR (PR+CR) according to RECIST (1.1), from the start of the treatment until PD/recurrence. The ORR in each treatment group is compared using Fisher's exact test. Exact confidence bounds (90% CI) are determined.

For patients in the Doxorubicin Arm who crossed over to olaratumab therapy, the best overall response is assessed separately for prior to and after the start of olaratumab therapy.

The duration of response for responders only is measured from the time measurement criteria are first met for CR/PR (whichever is first recorded) until the first date that the criteria for PD are met, or death. Duration of response is estimated with the Kaplan-Meier method; a 90% CI is provided for the median duration of response. Patients who do not relapse are censored at the day of their last objective tumor assessment.

### Phase 2 Interim Analysis Results

PFS: Interim analysis of the Combination Arm and Doxorubicin Arm (Table 2) showed a median PFS of 29.9 weeks (90% CI=23.7, 36.0) (95% CI=22.3, 36.7) and 17.9 weeks (90% CI=12.7, 23.3) (95% CI=12.1, 23.4) respectively. The stratified HR for this interim analysis was 0.597 (90% CI=0.415, 0.858) with a stratified Log-rank p-value of p=0.0184.

Further interim analysis of PFS of the Combination Arm and the Doxorubicin Arm was performed based on histological tumor type. Patients were categorized into 2 groups: leiomyosarcoma and other sarcomas. Other sarcomas include alveolar soft part sarcoma, chondroblastic osteosarcoma, chondrosarcoma, clear cell sarcoma, endometrial stromal sarcoma, epithelial sarcoma, epithelioid, extraskeletal myxoid chondrosarcoma, fibromyxoid sarcoma, fibrosarcoma, fibrosarcomatous transformation in a recurrent dermatofibrosarcoma, hemangiopericytoma, high grade undifferentiated sarcoma, liposarcoma, malignant fibrous histocytoma, malignant glomus tumor, malignant peripheral nerve sheath tumor, malignant solitary fibrous tumor, malignant spindle cell sarcoma, malignant spindle cell tumor with rhabdoid features, myxofibrosarcoma, myxoid chondrosarcoma, myxoid liposarcoma, myxoid sarcoma, neurofibrosarcoma, pleomorphic sarcoma (including: high grade spindle and pleomorphic sarcoma, of left thigh, poorly differentiated, round cell sarcoma, sarcoma, smooth muscle tumor, spindle cell sarcoma, undifferentiated), synovial sarcoma, undifferentiated sarcoma, undifferentiated uterine sarcoma.

Patients sub-typed with leiomyosarcoma showed a median PFS of 28.3 weeks (90% CI=15.0, 42.7) and 15.7 weeks (90% CI=7.1, 31.7) for the Combination and Doxorubicin Arms respectively. The HR for this interim analysis for the Combination Arm versus the Doxorubicin Arm for leiomyosarcoma was 0.671 (90% CI=0.377, 1.194). Patients sub-typed as “other sarcomas” showed a median PFS of 30.3 weeks (90% CI=22.3, 36.7) and 19.0 weeks (90% CI=10.3, 23.4) for the Combination and Doxorubicin Arms, respectively. The stratified HR for this interim analysis for the Combination Arm versus Doxorubicin Arm for “other sarcomas” was 0.608 (90% CI=0.395, 0.937), respectively.

OS: Interim analysis of the Combination Arm and the Doxorubicin Arm (Table 3) showed a median OS of 98.9 weeks (90% CI=89.6, NE) (95% CI=70.9, NE) and 58.7 weeks (90% CI=42.6, 78.1) (95% CI=40.1, 94.3), respectively. The stratified HR for this interim analysis was 0.460 (90% CI=0.288, 0.735) with a stratified Log-rank p=value of 0.0052. The 12-month OS rate and 18-month OS rate for the Combination Arm was 80.3% (90% CI=70.2, 87.3) and 64.2% (90% CI=50.9, 74.8), respectively. The corresponding 12-month and 18-month OS rate for the Doxorubicin Arm was 55.1% (90% CI=43.7, 65.1) and 40.0% (90% CI=27.5, 52.1), respectively.

Further interim analysis of OS of the Combination Arm and the Doxorubicin Arm was performed based on histological tumor type. Patients were categorized into 2 groups: leiomyosarcoma and “other sarcomas” as per the aforementioned list. Patients sub-typed with leiomyosarcoma showed an OS of 100.3 weeks (90% CI=89.6, NE) and 45.3 weeks (90% CI=40.1, 58.7) for the Combination and Doxorubicin Arms, respectively. The HR for this interim analysis for the Combination Arm versus the Doxorubicin Arm for leiomyosarcoma was 0.258 (90% CI=0.116, 0.570). Patients sub-typed with “other sarcomas” showed an OS of 98.9 weeks (90% CI=61.4, NE) and 78.1 weeks (90% CI=35.7, NE) for the Combination and Doxorubicin Arms respectively. The HR for this interim analysis for the Combination Arm versus the Doxorubicin Arm for “other sarcomas” was 0.716 (90% CI=0.399, 1.285), respectively.

ORR/Disease Control Rate: Interim analysis of response rate in the Combination Arm and Doxorubicin Arm (Table 4) showed disease control rate (CR+PR+SD) in 71.9% (90% CI=61.2, 81.0) and 53.8% (90% CI=42.9, 64.5) of patients respectively. The ORR (CR+PR) observed in the Combination Arm and the Doxorubicin Arm was 18.8% (90% CI=11.2, 28.6) with a p=0.2236 and 10.8% (90% CI=5.2, 19.3) respectively.

TABLE 2
PFS Interim Study Results of the
Combination and Doxorubicin Arms

| | Combination Arm | | |
|---|---|---|---|
| | (Olaratumab + | | |
| | Doxorubicin) | Doxorubicin Arm | Stratified |
| | Median Weeks | Median Weeks | Hazard Ratio |
| | (90% CI) | (90% CI) | (90% CI) |
| PFS | 29.9 weeks | 17.9 weeks | 0.597 |
| | (23.7, 36.0) | (12.7, 23.3) | (0.415, 0.858) |
| PFS | 28.3 weeks | 15.7 weeks | 0.671 |
| Leiomyosarcoma | (15.0, 42.7) | (7.1, 31.7) | (0.377, 1.194) |
| PFS | 30.3 weeks | 19.0 weeks | 0.608 |
| Other Sarcoma | (22.3, 36.7) | (10.3, 23.4) | (0.395, 0.937) |

TABLE 3
OS Interim Study Results of the
Combination and Doxorubicin Arms

| | Combination Arm | | |
|---|---|---|---|
| | (Olaratumab + | | |
| | Doxorubicin) | Doxorubicin Arm | Stratified |
| | Median Weeks | Median Weeks | Hazard Ratio |
| | (90% CI) | (90% CI) | (90% CI) |
| OS | 98.9 weeks | 58.7 weeks | 0.460 |
| | (89.6, NE) | (42.6, 78.1) | (0.288, 0.735) |
| OS | 100.3 weeks | 45.3 weeks | 0.258 |
| Leiomyosarcoma | (89.6, NE) | (40.1, 58.7) | (0.116, 0.570) |
| OS | 98.9 weeks | 78.1 weeks | 0.716 |
| Other Sarcoma | (61.4, NE) | (35.7, NE) | (0.399, 1.285) |

NE = the limit was not reached

TABLE 4
Disease Control and ORR for Study Results
of the Combination and Doxorubicin Arms

| | Combination Arm | |
|---|---|---|
| | (Olaratumab + | |
| | Doxorubicin) | Doxorubicin Arm |
| Disease Control Rate | 71.9% | 53.8% |
| (CR + PR + SD) (%) | (61.2, 81.0) | (42.9, 64.5) |
| (90% CI) | | |
| ORR | 18.8% | 10.8% |
| (CR + PR) (%) | (11.2, 28.6) | (5.2, 19.3) |
| (90% CI) | | |

The interim analysis of the clinical data from the Study over the spectrum of subtypes, illustrate 29.9 weeks of median PFS which is a 12-week improvement in PFS when olaratumab is combined with doxorubicin as compared to doxorubicin alone (see Table 2). This 12-week improvement in PFS is particularly unexpected based on the design of the Study wherein the Study was designed to be considered successful with a 4-week PFS improvement in the Combination Arm over the Doxorubicin Arm.

The interim analysis of the clinical data from the Study over the spectrum of subtypes also shows a median OS of 98.9 weeks which is a 40-week improvement of OS when olaratumab is combined with doxorubicin as compared to doxorubicin alone (see Table 3). A 40-week improvement of OS is highly unexpected.

Additionally, from a statistical perspective, phase 2 trials are typically designed to illustrate PFS, not OS, in part, due to the limited sample size of the Study; phase 2 trials are typically not designed to demonstrate statistically significant evidence of an OS benefit and certainly not of the magnitude demonstrated in the Study. A relatively small population will not show statistical significance unless a remarkable difference is observed. It is unexpected for the data to show a clear statistical improvement in the OS in a phase 2 study; the interim analysis of the clinical data from the Study, over the spectrum of subtypes, demonstrates a similar OS benefit trend.

When the PFS and OS of the Study are compared to the currently available and recently investigated treatments, significant improvements over the current standard of care in terms of (1) a lower hazard ratio and (2) a larger improvement in median survival time, illustrate a clear unexpected benefit.

Finally, according to the interim analysis, the toxicity profile of the combination of olaratumab combined with doxorubicin is overall acceptable and well tolerated as compared to other STS treatments with regard to adverse events.

### Phase 2 Final Analysis Results

Final results from the primary analysis substantially confirmed the trends seen in the interim analysis.

Antitumor Effects of Olaratumab in Combination with or without Doxorubicin in Murine Xenograft Models for SKLMS-1 Leiomyosarcoma, KHOS/NP Osteosarcoma and A204 Rhabdomysarcoma.

### SKLMS-1 Leiomyosarcoma Xenograft Model

6 to 7-week-old female nu/nu athymic mice are injected with 5×10⁶ SKLMS-1 (ATCC #HBT-88) cells/mouse in 50% Matrigel (BD Biosciences #356235). When tumors are approximately 300 mm³ in volume, the mice are randomized by tumor volume into 4 treatment groups (n=12): Group 1 control mice are dosed with USP Saline (Aldwin Scientific #2F7124) at 10 μL/gram, Group 2 mice are dosed with olaratumab at 60 mg/kg (loading dose 214 mg/kg), Group 3 mice are dosed with doxorubicin (Sigma #D-1515) at 3 mg/kg and Group 4 mice are dosed with a combination of olaratumab and doxorubicin at the monotherapy concentrations of 60 mg/kg olaratumab and 3 mg/kg doxorubicin. The combinations are dosed so that olaratumab is administered 1-2 hours prior to doxorubicin on the days of treatment. All treatments are administered via intra-peritoneal injection twice a week on Tuesday and Friday.

Olaratumab and doxorubicin are prepared in USP Saline. Tumor volume is measured by three dimensional caliper measurements twice a week during the course of the study and calculated as volume in mm³=L (longer of measured dimension)×W² (shorter dimension)×(pi/6). A repeated measures ANOVA is used to evaluate the differences in tumor growth and body weight between the treatment groups. Relative changes in tumor volume (% T/C) are calculated using the tumor volume measurements taken at day 21 and the baseline tumor volume is the volume recorded on or just prior to first day of dosing.

Results: Combination treatment of olaratumab and doxorubicin resulted in a statistically significant improvement in antitumor efficacy as compared to each of the monotherapy groups. Tumor volume at day 21 showed a % T/C of 46% (p<0.0001) in the combination group as compared to a % T/C of 64% in both the respective monotherapy groups. When compared to the saline control group, these values were determined to be statistically significant with p<0.0001, p=0.0015 and p=0.001, respectively, for the combination, olaratumab monotherapy and doxorubicin monotherapy treatment groups.

The preclinical data in the SKLMS-1 leiomyosarcoma xenograft model further support the efficacy of the combination of the olaratumab and doxorubicin in STS with respect to reduction in tumor volume as compared to the use of either olaratumab or doxorubicin as a monotherapy.

### KHOS/NP Osteosarcoma Xenograft Model

6 to 7-week-old female nu/nu athymic mice are injected with 1×10⁶ KHOS/NP (ATCC #CRL-1544) cells/mouse in 50% Matrigel (BD Biosciences #356235). When tumors are approximately 450 mm³ in volume, the mice are randomized by tumor volume into 4 treatment groups (n=12): Group 1 control mice are dosed with USP Saline (Aldwin Scientific #2F7124) at 10 μL/gram, Group 2 mice are dosed with olaratumab at 60 mg/kg (loading dose 214 mg/kg), Group 3 mice are dosed with doxorubicin (Sigma #D-1515) at 3 mg/kg and Group 4 mice are dosed with a combination of olaratumab and doxorubicin at the monotherapy concentrations of 60 mg/kg olaratumab and 3 mg/kg doxorubicin. The combinations are dosed so that doxorubicin is administered 3 hours prior to olaratumab on the days of treatment. All treatments are administered via intra-peritoneal injection twice a week on Monday and Thursday.

Olaratumab and doxorubicin are prepared in USP Saline. Tumor volume is measured by three dimensional caliper measurements twice a week during the course of the study and calculated as volume in mm³=L (longer of measured dimension)×W² (shorter dimension)×(pi/6). A repeated measures ANOVA is used to evaluate the differences in tumor growth and body weight between the treatment groups. Relative changes in tumor volume (% T/C) are calculated using the tumor volume measurements taken at day 32, whereas the baseline tumor volume is the volume recorded on or just prior to first day of dosing.

Results: Combination treatment of olaratumab and doxorubicin resulted in a statistically significant improvement in antitumor efficacy as compared to each of the monotherapy treatment groups. Tumor volume at day 32 showed a % T/C of 55% in the combination group as compared to a % T/C of 79% and % T/C of 72% in the olaratumab and doxorubicin monotherapy groups, respectively. When compared to the saline control group, these values were determined to be statistically significant with p<0.0001, p=0.01 and p=0.002, respectively for the combination, olaratumab monotherapy and doxorubicin monotherapy treatment groups.

The preclinical data in the KHOS/NP osteosarcoma xenograft model further support the efficacy of the combination of the olaratumab and doxorubicin in sarcoma with respect to reduction in tumor volume as compared to the use of either olaratumab or doxorubicin as a monotherapy.

### A204 Rhabdoid Xenograft Model

The A204 model was originally classified as RMS, but has since been determined to be more likely rhabdoid. Hinson, Ashley R. P. et al. Frontiers in Oncology 3 (2013): 183. PMC. Web. 8 May 2015. Rhabdoid tumors are of unknown origin and are rare. Currently, it is believed that malignant rhabdoid tumors are kidney in origin, but the exact cell of origin remains unknown.

7 to 8-week-old female nu/nu athymic mice are injected with 5×10⁶ A204 (ATCC #CRL-7900) cells/mouse in 100% Matrigel (BD Biosciences #356235). When tumors are approximately 340 mm³ in volume, the mice are randomized by tumor volume into 2 treatment groups (n=12): Group 1 control mice are dosed with HuIgG (Equitech-Bio #SLH66-0001) at 40 mg/kg and Group 2 mice are dosed with olaratumab at 40 mg/kg. All treatments are administered via intra-peritoneal injection 3×a week on Monday, Wednesday and Friday.

Olaratumab is diluted in USP Saline (Aldwin Scientific #2F7124) at 4 mg/mL. Tumor volume is measured by three dimensional caliper measurements twice a week during the course of the study and calculated as volume in mm³=L (longer of measured dimension)×W² (shorter dimension)×(pi/6). A repeated measures ANOVA is used to evaluate the differences in tumor growth and body weight between the treatment groups. Relative changes in tumor volume (%T/C) are calculated using the tumor volume measurements taken at day 30, whereas the baseline tumor volume is the volume recorded on or just prior to first day of dosing.

Results: Olaratumab treatment when compared to the HuIgG control group showed a statistically significant improvement (p<0.0001) in antitumor efficacy with a % T/C of 37%.

The preclinical data in the A204 rhabdoid tumor xenograft model supports the efficacy of olaratumab in rhabdoid tumors with respect to the reduction of tumor volume as compared to saline.

### Additional Sequences

SEQ ID NO. 11
atgggatggtcatgtatcatcattactagtagcaactgcaactggagta
cattcacagctgcagctgcaggagtcgggcccaggactggtgaagcctt
cggagaccctgtccctcacctgcactgtctctggtggctccatcaacag
tagtagttactactggggctggctccgccagtccccagggaaggggctg
gagtggattgggagatcattatactgggagcacctactacaacccgtcc
ctcaggagtcgactcaccatatccgtagacacgtccaagaaccagactc
cctgatgctgagactgtgaccgccgcagacacggctgtatattactgtg
cgagacagtccacgtattactatggttcggggaattattatggctggtt
cgaccgctgggaccagggaaccctggtcaccgtctcctcagctagcacc
aagggcccatcggtatccccctggcaccctcctccaagagcacctctgg
gggcacagcggccctgggctgcctggtcaaggactacttccccgaaccg
gtgacggtgtcgtggaactcaggcgccctgaccagcggcgtgcacacct
tcccggctgtcctacagtcctcaggactctactccctcagcagcgtggt
gaccgtgccctccagcagcagggcacccagacctacatctgcaacgtga
atcacaagcccagcaacaccaaggtggacaagagagttgagcccaaatc
agtgacaaaactcacacatgcccaccgtgcccagcacctgaactcctgg
ggggaccgtcagtatcctatccccccaaaacccaaggacaccctcatga
tctcccggacccctgaggtcacatgcgtggtggtggacgtgagccacga
agaccctgaggtcaagttcaactggtacgtggacggcgtggaggtgcat
aatgccaagacaaagccgcgggaggagcagtacaacagcacgtaccgtg
tggtcagcgtcctcaccgtcctgcaccaggactggctgaatggcaagga
gtacaagtgcaaggtctccaacaaagccctcccagcccccatcgagaaa
accatctccaaagccaaagggcagccccgagaaccacaggtgtacaccc
tgcccccatcccgggaggagatgaccaagaaccaggtcagcctgacctg
cctggtcaaaggcactatcccagcgacatcgccgtggagtgggagagca
atgggcagccggagaacaactacaagaccacgcctcccgtgctggactc
cgacggctcatatcctctatagcaagctcaccgtggacaagagcaggtg
gcagcaggggaacgtcttctcatgctccgtgatgcatgaggctctgcac
aaccactacacgcagaagagcctctccctgtccccgggtaaatga
SEQ ID NO. 12
atgggatggtcatgtatcatcattactagtagcaactgcaactggagta
cattcagaaattgtgagacacagtctccagccaccctgtctagtctcca
ggggaaagagccaccctctcctgcagggccagtcagagtgttagcagct
acttagcctggtaccaacagaaacctggccaggctcccaggctcctcat
ctatgatgcatccaacagggccactggcatcccagccaggttcagtggc
agtgggtctgggacagacttcactctcaccatcagcagcctagagcctg
aagattagcagatattactgtcagcagcgtagcaactggcctccggcgt
tcggccaagggaccaaggtggaaatcaaacgtacggtggctgcaccatc
tgtatcatcacccgccatctgatgagcagttgaaatctggaactgcctc
tgagtgtgcctgctgaataacactatcccagagaggccaaagtacagtg
gaaggtggataacgccctccaatcgggtaactcccaggagagtgtcaca
gagcaggacagcaaggacagcacctacagcctcagcagcaccctgacgc
tgagcaaagcagactacgagaaacacaaagtctacgcctgcgaagtcac
ccatcagggcctgagctcgcccgtcacaaagagcttcaacaggggagag
tgttag

1-17. (canceled)

18. A method of treating soft tissue sarcoma, comprising:
administering olaratumab to a patient, wherein the patient has soft tissue sarcoma;
administering a cardioprotective agent to the patient, wherein the cardioprotective agent is administered following said step of administering olaratumab; and
administering doxorubicin to the patient, wherein doxorubicin is administered following said step of administering the cardioprotective agent.

19. The method according to claim 18, wherein the olaratumab is administered at a dose of about 15 mg/kg.

20. The method according to claim 18, wherein the doxorubicin is administered at a dose of about 60 mg/m² or about 75 mg/m².

21. The method according to claim 20, wherein the doxorubicin is administered at a dose of about 75 mg/m².

22. The method according to claim 18, wherein the cardioprotective agent is dexrazoxane.

23. The method according to claim 18, wherein the olaratumab is administered at a dose of about 15 mg/kg, wherein the doxorubicin is administered at a dose of about 75 mg/m², and wherein the cardioprotective agent is dexrazoxane and is administered at a dose of about 750 mg/m².

24. The method according to claim 18, wherein the soft tissue sarcoma is leiomyosarcoma.

25. A method of treating soft tissue sarcoma, comprising:
administering olaratumab to a patient, wherein the patient has soft tissue sarcoma; and
administering doxorubicin to the patient,
wherein following a fourth administration of doxorubicin to the patient, a cardioprotective agent is administered to the patient, wherein the cardioprotective agent is administered following administration of olaratumab to the patient and prior to administration of doxorubicin to the patient.

26. The method according to claim 25, wherein the olaratumab is administered at a dose of about 15 mg/kg.

27. The method according to claim 25, wherein the doxorubicin is administered at a dose of about 60 mg/m² or about 75 mg/m².

28. The method according to claim 27, wherein the doxorubicin is administered at a dose of about 75 mg/m².

29. The method according to claim 25, wherein the cardioprotective agent is dexrazoxane.

30. The method according to claim 25, wherein the olaratumab is administered at a dose of about 15 mg/kg, wherein the doxorubicin is administered at a dose of about 75 mg/m², and wherein the cardioprotective agent is dexrazoxane and is administered at a dose of about 750 mg/m².

31. The method according to claim 25, wherein the soft tissue sarcoma is leiomyosarcoma.

32. A pharmaceutical composition comprising olaratumab with one or more pharmaceutically acceptable carriers, diluents, or excipients, in combination with a pharmaceutical composition of doxorubicin with one or more pharmaceutically acceptable carriers, diluents, or excipients, in combination with a pharmaceutical composition of a cardioprotective agent with one or more pharmaceutically acceptable carriers, diluents, or excipients, wherein the cardioprotective agent is administered to the patient after the olaratumab is administered and before the doxorubicin is administered.

33. The pharmaceutical composition according to claim 32, wherein the olaratumab is administered at a dose of about 15 mg/kg.

34. The pharmaceutical composition according to claim 32, wherein the doxorubicin is administered at a dose of about 60 mg/m² or about 75 mg/m².

35. The pharmaceutical composition according to claim 34, wherein the doxorubicin is administered at a dose of about 75 mg/m².

36. The pharmaceutical composition according to claim 32, wherein the cardioprotective agent is dexrazoxane.

37. The pharmaceutical composition according to claim 32, wherein the olaratumab is administered at a dose of about 15 mg/kg, wherein the doxorubicin is administered at a dose of about 75 mg/m², and wherein the cardioprotective agent is dexrazoxane and is administered at a dose of about 750 mg/m².

38. The pharmaceutical composition according to claim 32, wherein the pharmaceutical composition is used for the treatment of soft tissue sarcoma.

39. The pharmaceutical composition according to claim 38, wherein the soft tissue sarcoma is leiomyosarcoma.
